(19) United States
(12) Patent Application Publication
(10) Pub. No.: US 2012/0323019 A1
(43) Pub. Date: Dec. 20, 2012

(54) METHOD FOR CARBOXYLIZING AROMATES AND HETETOAROMATES USING CO2

(75) Inventors: Christian Severins, Koln (DE); Sigurd Buchholz, Koln (DE); Kilian Tellman, Frankfurt am Main (DE); Jürgen Wieschemeyer, Bergisch Gladbach (DE); Klaus Weidemann, Wuppertal (DE)

(73) Assignee: BAYER INTELLECTUAL PROPERTY GMBH, Monheim (DE)

(21) Appl. No.: 13/517,019

(22) PCT Filed: Dec. 17, 2010
(86) PCT No.: PCT/EP2010/070051

§ 371 Date: Aug. 21, 2012

(30) Foreign Application Priority Data
Dec. 21, 2009 (DE) 10 2009 060 033.7

(51) Int. Cl. C07D 333/40; C07D 307/68

(52) U.S. Cl. 549/71; 549/484

(57) ABSTRACT

The present invention relates to a process for the preparation of aromatic and heteroaromatic carboxylic acids using CO₂.

The present invention relates to a process for the preparation of aromatic and heteroaromatic carboxylic acids using CO₂.

Aromatic and heteroaromatic carboxylic acids have been known for a long time. They are inter alia building blocks of many pharmaceutical products and their selective synthesis is therefore of decisive importance. One example of a heteroaromatic carboxylic acid which may be mentioned is thiophene-2-carboxlic acid, the derivatives of which have a microbicidal effect.

There are numerous different preparation methods for aromatic and heteroaromatic carboxylic acids. Virtually all methods are multi-stage. A two-stage process consisting of an acylation of the aromatics or heteroaromatics with subsequent oxidation to give the corresponding carboxylic acids is particularly widespread. Usually, the corresponding Friedel-Crafts acylations are carried out in the presence of stoichiometric amounts of Lewis acids in anhydrous solvents (see e.g. DE 102007032451A1, EP178184A1).

Transferring such reactions from the laboratory to an industrial scale always presents a considerable problem since the solvents are environmentally-burdensome in a different way. During the product isolation, relatively large amounts of wastewaters with a high salt content are also produced and these have to be worked-up. The oxidation of the aryl ketone is usually carried out with organic peroxides or inorganic oxidizing agents (Dodd et al. Synthesis 1993, 295-297; U.S. Pat. No. 5,739,352). Transferring such oxidations to the industrial scale likewise represents a considerable problem since the oxidizing agents are environmentally-burdensome in a different way and the reactions are highly exothermic.

An efficient synthesis method for aromatic and heteroaromatic carboxylic acids is a so-called direct carboxylation with CO₂. Moreover, CO₂ is a nontoxic and readily available, cost-effective C₁ source. Nevertheless, there are only a few literature examples of the direct carboxylation of aromatics and heteroaromatics with CO₂.

U.S. Pat. No. 2,948,737 describes such a direct carboxylation of heteroaromatics. It is disclosed therein that the direct carboxylation with gaseous CO₂ is possible at temperatures >300° C. in the presence of acid-binding reagents at a reaction pressure of 1570 bar in an autoclave with moderate yields (8%).

U.S. Pat. No. 3,138,626 describes that the direct carboxylation with gaseous CO₂ can be carried out above temperatures of 100° C. in the presence of AlCl₃ at a reaction pressure of 200 bar in an autoclave with moderate yields (22%).

Transferring such reactions to the industrial scale represents a considerable problem on account of the high reaction temperatures since many carboxylic acids of aromatics and heteroaromatics have considerably lower decomposition temperatures.

Ohishi et al. (Angew. Chem Int. Ed. 2008, 47, 5792-5795) describe experiments in which aromatic and heteroaromatic carboxylic acids have been prepared in organic solvents using mixtures consisting of boronic acid esters, a homogeneous copper-carbene catalyst and CO₂ at significantly lower temperatures (70° C.).

Oshima et al. (Org. Lett., 2008, 10, 2681-2683) disclose experiments in which aromatic carboxylic acids have been prepared at room temperature using mixtures consisting of organic zinc compounds, a homogeneous nickel-phosphorus catalyst and gaseous CO₂.

One problem when transferring these reactions to an industrial process is the use of cost-intensive homogeneous catalysts, which cannot be recycled. During the product isolation, relatively large amounts of wastewaters with a high salt content are also produced; these have to be worked-up.

There is accordingly a need for a cost-effective process that can be carried out easily for preparing aromatic and heteroaromatic carboxylic acids which can also be carried out on the industrial scale.

Proceeding from the known prior art, the technical object is therefore to provide a process for the preparation of aromatic and heteroaromatic carboxylic acids which is comparatively simple to carry out and is cost-effective and leads to higher yields. The desired process should also have the lowest possible environmental hazard potential and a safe temperature control. The formation of large amounts of salt-like wastewaters should be avoided. In particular, it should be possible to use the process for the carboxylation of thiophene and/or furan and/or derivatives thereof.

According to the invention, this object is achieved by a process according to Claim 1. Preferred embodiments can be found in the dependent claims.

The process according to the invention for the carboxylation of aromatics and heteroaromatics comprises at least the following steps:
a) provision of a first liquid component comprising an aromatic and/or heteroaromatic compound,
b) provision of a second liquid component comprising an organic and/or inorganic base,
c) mixing of the first and second liquid components,
d) mixing of the mixture from step c) with CO₂ with reaction of the aromatic or heteroaromatic compound with CO₂.

In step a) of the process according to the invention, a first liquid component, at least comprising one aromatic compound and/or one heteroaromatic compound, is provided. An aromatic compound is also abbreviated here to aromatic and a heteroaromatic compound is abbreviated to heteroaromatic.

One or more aromatics and/or heteroaromatics are used as starting material and carboxylated in the process according to the invention.

The starting material is provided in liquid form. In this connection, the starting material (aromatic, heteroaromatic) can already be present in liquid form. In this case, the component referred to in step a) as first liquid component can be the liquid starting material. It is likewise conceivable to dissolve the starting material firstly in a solvent and to provide this solution as first liquid component.

Aromatics and heteroaromatics are understood as meaning organic compounds which have a planar, cyclic structural motif of conjugated double bonds and/or free electron pairs or unoccupied p orbitals.

In the conjugated double bonds, comparatively low energy levels are present for the bonding electrons, for which reason conjugated double bonds are identified by reduced and altered reactivity compared with other (nonconjugated) double-bond systems.

Whereas the cyclic structural motif of aromatics is formed only by carbon atoms, heteroaromatics have one or more heteroatoms, i.e. non-carbon atoms in the ring structure, e.g. oxygen, nitrogen and/or sulphur.

Aromatic or heteroaromatic compounds which can be used are benzene derivatives, particularly with heteroatoms in the side chain such as anisole or dimethylaniline, six-membered heteroaromatics such as pyridine, five-membered heteroaromatics such as pyrrole, seven-membered aromatics such as azepine, thiepine, oxepine.

Preference is given to using heteroaromatics which have one or more heteroatoms which function as π electron donor and increase the electron density within the ring.

Preference is given to using aromatics and/or heteroaromatics which have a five-membered ring since here the electron density is increased compared to a six-membered ring.

In the process according to the invention, very particular preference is given to using thiophene and/or furan and/or derivatives of thiophene and/or furan. A derivative is understood as meaning a chemical compound which can be derived from a basic substance (here, for example furan or thiophene). A derivative is characterized in that there is another atom or another atom group at at least one point in the molecule of the basic substance.

Carboxylation is understood as meaning the introduction of a carboxyl group into an organic compound. Carboxylation is a reaction for producing carboxylic acids.

In step b) of the process according to the invention, a second liquid component at least comprising an organic and/or inorganic base is provided. The second liquid component can be the base itself; it is likewise conceivable that the second liquid component is a solution in which an organic and/or inorganic base is present.

Preferably, the base used is n-butyllithium, t-butyllithium, methyllithium, phenyllithium, lithium diisopropylamide (LDA) and/or hexyllithium.

In step c) of the process according to the invention, a mixing of the first and second liquid components takes place.

The combining of the liquid components takes place preferably at a temperature in the range from −100° C. to 40° C. and at a pressure of from 1 to 60 bar.

It is the aim of step c) to produce as homogeneous a mixture as possible of the two liquid components.

In step d) of the process according to the invention, the mixing of the mixture obtained from step c) with CO₂ takes place. CO₂ can be added in gaseous, liquid, solid or supercritical state or in solution to the mixture of the base and the aromatic and/or heteroaromatic. Preferably, the addition of CO₂ takes place in the gaseous or liquid state.

The mixing in step d) takes place preferably at a temperature in the range from −100° C. to 60° C. and at a pressure of from 1 to 60 bar.

The carboxylation of the aromatic and/or heteroaromatic is initiated with the addition of CO₂. The reaction between the aromatic and/or the heteroaromatic with CO₂ is carried out up to the desired or achievable conversion.

After reacting the reactants, the reaction mixture is preferably worked-up in order to isolate, and optionally to purify, the desired carboxylated product. The process according to the invention therefore preferably comprises a further step e) following step d):

e) capture of the mixture from step d) and isolation of the carboxylated product.

To isolate the carboxylated aromatic or heteroaromatic, the reaction mixture is preferably firstly provided with acid in order to bind amounts of base that are still present. The carboxylated product can be isolated for example by extraction and/or distillation and/or chromatography.

The process according to the invention can be carried out continuously or discontinuously. It is likewise conceivable to carry out some steps of the process according to the invention continuously and the other steps discontinuously. Preferably, at least steps c) and d) are carried out continuously. Continuous steps for the purposes of the invention are those in which the feed of compounds (starting materials) into a reactor and the discharge of compounds (products) from the reactor take place simultaneously but spatially separately, whereas in the case of discontinuous steps the sequence feed of compounds (starting materials), optional chemical reaction and discharge of compounds (products) proceed successively. The continuous procedure is economically advantageous since reactor down-times as a consequence of filling and emptying processes and long reaction times on account of safety precautions, reactor-specific heat exchange performances and also heating and cooling processes, as arise in the case of batch processes (discontinuous processes), are avoided.

The preferably continuous mixing of compounds in step c) and/or in step d) is preferably carried out by means of a static mixer.

Whereas in the case of dynamic mixers the homogenization of a mixture is achieved by means of agitated elements such as e.g. stirrers, in the case of static mixers, the flow energy of the fluid is utilized: a conveying unit (e.g. a pump) forces the liquid e.g. through a tube provided with static mixing internals, where the liquid following the main flow axis is divided into part streams which, depending on the type of internals, are swirled with one another and mixed.

An overview of different types of static mixers as used in conventional process technology is given for example in the article “Statische Mischer and ihre Anwendungen [Static mixers and their applications]”, M. H. Pahl and E. Muschelknautz, Chem.-Ing.-Techn. 52 (1980) No. 4, pp. 285-291.

An example of static mixers which may be mentioned here is SMX mixers (cf. patent specification U.S. Pat. No. 4,062,524). They consist of two or more grids which are perpendicular to one another and are composed of parallel strips which are joined together at their intersections and are set at an angle with respect to the main flow direction of the mixing material in order to divide the liquid into part streams and to mix them. An individual mixing element is unsuitable as mixer since thorough mixing takes place only along a preferential direction transverse to the main flow direction.

Consequently, a plurality of mixing elements, rotated by 90° relative to one another, therefore have to be arranged one after another.

For the process according to the invention or for steps of the process according to the invention, the use of microprocess technology is advantageous.

Modular microprocess technology or microreaction technology offers the possibility of combining different microprocess modules in the manner of building blocks to form a complete production plant in a very small format.

Modular microreaction systems are supplied commercially, e.g. by Ehrfeld Mikrotechnik BTS GmbH. The commercially available modules include mixers, reactors, heat exchangers, sensors and actuators and many more.

Preferably, the mixing in step c) and/or step d) takes place by means of one or more so-called micromixers.

The term “micromixer” used is here representative of microstructured, preferably continuously operating reactors which are known under the term microreactor, minireactor, micro heat exchanger, minimixer or micromixer. Examples are microreactors, micro heat exchangers, T- and Y-mixers, and also micromixers from a wide variety of companies (e.g. Ehrfeld Mikrotechnik BTS GmbH, Institut für Mikrotechnik Mainz GmbH, Siemens AG, CPC-Cellulare Process Chemistry Systems GmbH, and others), as are generally known to the person skilled in the art, where a “micromixer” for the purposes of the present invention usually has characteristic/determining internal dimensions of up to 1 mm and contains static mixing internals. An example of a static micromixer which may be mentioned is the faceted mixer described in DE20219871U1.

By reducing the characteristic dimensions, besides heat transfer processes, mixing processes also proceed considerably more quickly in micromixers than in conventional mixers. Thus, the processing speeds in micromixers are sometimes several powers of ten higher than in conventional apparatuses, and the mixing sections are reduced to a few millimetres.

Preferably, the reaction of an aromatic and/or heteroaromatic in step d) of the process according to the invention is carried out by passing the reaction mixture through a residence section. Preferably, the residence section has one or more static mixers.

The metering rate of all components and the flow rate of the reaction mixture through the residence section depend primarily on the desired residence times and/or conversions to be achieved. The higher the maximum reaction temperature, the shorter the residence time should be. As a rule, the reactants in the reaction zone have residence times between 20 seconds (20 sec) and 400 minutes (400 min), preferably between 1 min and 400 min, very particularly preferably between 1 min and 20 min.

The residence time can be controlled for example via the volume streams and the volume of the reaction zone. The course of the reaction is advantageously monitored by means of various measuring devices. Of suitability for this purpose are in particular devices for measuring the temperature, the viscosity, the thermal conductivity and/or the refractive index in flowing media and/or for measuring infrared and/or near-infrared spectra.

It is conceivable to feed CO₂ into the reaction mixture along part of the residence section or along the entire residence section.

The process according to the invention can preferably be carried out in heatable flow reactors. In a preferred embodiment, the reaction plant for carrying out the process according to the invention comprises at least two zones which can be heated independently of one another. In the first zone, the mixing of the liquid components, comprising an aromatic and/or heteroaromatic compound and an inorganic and/or organic base, takes place (step c)). In the second zone, the reaction zone, the addition of CO₂ and the reaction of the aromatic and/or heteroaromatic compound takes place (step d)). At the end of the reaction zone, the product is preferably captured and collected in order to isolate the desired product in a downstream step (step e)).

The invention is explained in more detail below by reference to examples without, however, limiting it thereto.

### EXAMPLE 1

### Preparation of 5-chlorothiophene-2-carboxylic acid by direct carboxylation with CO₂

A solution of 12.5 mass fractions of 2-chlorothiophene and 87.5 mass fractions of THF was poured into receiver 1. A solution of 23 mass fractions of n-butyllithium and 77 mass fractions of hexane were poured into receiver 2. The two receivers were connected via a preheating section (0° C.) to a static mixer (volume 0.3 ml), to the outlet channel of which a residence element with a volume of 4.3 cm³ and a ratio of area to volume of 22.6 cm²/cm⁻³ (0° C.) was attached and led to an inlet of a further static mixer (volume 0.3 ml). Attached to the second inlet of the static mixer was, via a pressure-reducing valve (1.3 bar), a CO₂ gas bottle, to whose outlet channel a residence element with a volume of 0.9 cm³ and a ratio of area to volume of 40 cm²/cm⁻³ (0° C.) was attached. The solution from receiver 1 was pumped continuously through the reactor at a volume rate of 56 ml/h and the solution from receiver 2 was pumped continuously through the reactor with a volume flow rate of 24 ml/h. The total residence time was 3.5 min. The reaction was monitored regularly by HPLC. The relative yield of 5-chlorothiophene-2-carboxylic acid was >70%. The product stream was quenched at 0° C. on 5.7M HCl solution. Following phase separation and washing of the aqueous phase with n-hexane, the combined organic phases were concentrated to dryness. The 5-chlorothiophene-2-carboxylic acid was taken up, for the purposes of purification, in a solvent mixture of 50 mass fractions of hexane, 35 mass fractions of methanol and 15 mass fractions of water. The aqueous phase was then concentrated in vacuo in order to remove the methanol. 5-Chlorothiophene-2-carboxylic acid crystallizes after cooling the mother liquor to 5° C. in the form of white needles.

### EXAMPLE 2

### Preparation of 2-furoic acid by direct carboxylation with CO₂

A solution of 5 mass fractions of furan and 95 mass fractions of THF was poured into receiver 1. A solution of 23 mass fractions of n-butyllithium and 77 mass fractions of hexane were poured into receiver 2. The two receivers were connected via a preheating section (−30° C.) to a static mixer (volume 0.3 ml), to the outlet channel of which a residence element with a volume of 5.4 cm³ and a ratio of area to volume of 26.3 cm²/cm⁻³ (−30° C.) was attached and led to an inlet of a further static mixer (volume 0.3 ml). Attached to the second inlet of the static mixer was, via a pressure-reducing valve (1.3 bar), a CO₂ gas bottle, to whose outlet channel a residence element with a volume of 3.8 cm³ and a ratio of area to volume of 18.2 cm²/cm⁻³ (0° C.) was attached. The solution from receiver 1 was pumped continuously through the reactor at a volume flow rate of 139 ml/h and the solution from receiver 2 was pumped continuously through the reactor with a volume flow rate of 40 ml/h. The total residence time was 2.0 min. The reaction was monitored regularly by HPLC. The relative yield of 2-furoic acid was >80%. The product stream was quenched at 0° C. on 5.7M HCl solution. Following phase separation and washing of the aqueous phase with n-hexane, the combined organic phases were concentrated to dryness. The 5-chlorothiophene-2-carboxylic acid was taken up, for the purposes of purification, in a solvent mixture of 50 mass fractions of hexane, 35 mass fractions of methanol and 15 mass fractions of water. The aqueous phase was then concentrated in vacuo in order to remove the methanol. 2-Furoic acid crystallizes after cooling the mother liquor to 5° C. in the form of yellowish needles.

1. A process for carboxylation of an aromatic and/or heteroaromatic comprising at least:
a) providing a first liquid component comprising an aromatic and/or heteroaromatic compound,
b) providing a second liquid component comprising an organic and/or inorganic base,
c) mixing the first and second liquid components,
d) mixing the first and second liquid components from c) with CO₂ and reacting the aromatic or heteroaromatic compound with CO₂.

2. The process according to claim 1, further comprising:
e) capturing a resultant mixture from d) and isolating a carboxylated product.

3. The process according to claim 1, comprising continuously carrying out c) and/or d).

4. The process according to claim 1, comprising carrying out c) and/or d) by a static mixer.

5. The process according to claim 1, comprising carrying out said reacting of CO₂ with an aromatic and/or heteroaromatic compound in a microreaction plant.

6. The process according to claim 1, wherein said aromatic and/or heteroaromatic compound is at least one compound selected from the group consisting of: derivatives of benzene, optionally with heteroatoms in the side chain optionally anisole or dimethylaniline, six-membered heteroaromatics optionally pyridine, five-membered heteroaromatics optionally pyrrole, thiophene or furan, and derivatives of these compounds, and seven-membered aromatics optionally azepine, thiepine or oxepine.

7. The process according to claim 1, wherein the inorganic and/or organic base is at least one compound selected from the group consisting of: n-butyllithium, t-butyllithium, methyllithium, phenyllithium, lithium diisopropylamide (LDA) and hexyllithium.

8. The process according to claim 1, wherein CO₂ is added in a gaseous and/or liquid state.

9. The process according to claim 1, wherein a resultant reaction mixture in step d) is passed through a residence section, having at least one static mixer.

10. The process according to claim 9, wherein said reaction mixture in step d) spends a residence time in the range from 20 seconds to 400 minutes in said residence section.
